(19) United States
(12) Patent Application Publication
(10) Pub. No.: US 2007/0238959 A1
(43) Pub. Date: Oct. 11, 2007

(54) Method and device for visualizing 3D objects

(75) Inventors: Matthias John, Nurnberg (DE); Marcus Pfister, Bubenreuth (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/656,789

(22) Filed: Jan. 23, 2007

(30) Foreign Application Priority Data
Jan. 23, 2006 (DE) 10 2006 003 126.1

(51) Int. Cl. G01N 23/04; A61B 5/05

(52) U.S. Cl. 600/407; 600/425; 378/62

(57) ABSTRACT

The present invention relates to a method and a device for visualizing three dimensional objects, in particular in real time. A three dimensional image data set of the object is created and registered with recorded two dimensional transillumination images of the object. For visualization purposes the edges of the object are extracted from the three dimensional data set and visually combined with the two dimensional transillumination images containing the edges of the object.

### CROSS REFERENCE TO RELATED APPLICATIONS

This application claims priority of German application No. 10 2006 003 126.1 filed Jan. 23, 2006, which is incorporated by reference herein in its entirety.

### FIELD OF THE INVENTION

The present invention relates to a method and a device for visualizing three dimensional objects, in particular in real time. The method and the device are particularly suitable for visualizing three dimensional objects during surgical operations.

### BACKGROUND OF THE INVENTION

For the purpose of navigating surgical instruments during a surgical operation, for example on the head or the heart, real time images are obtained with the aid of fluoroscopic transillumination. Compared with three dimensional angiographic images, these transillumination images show absolutely no spatial, that is, three dimensional details, even though they are available in real time and minimize the radiation load for both patient and surgeon.

In order to supplement the two dimensional transillumination images with spatial information, the two dimensional transillumination images are registered with and combined with preoperatively recorded three dimensional images. The preoperatively recorded three dimensional images can be created by the classic medical imaging methods such as computed tomography (CT), three dimensional angiography, three dimensional ultrasound, positron emission tomography (PET) or magnetic resonance tomography (MRT).

The registration and superimposition of the two dimensional transillumination images with the previously recorded three dimensional images then provide the surgeon with improved guidance in the volume.

There are now two steps involved in the registration and superimposition of the two dimensional and three dimensional images.

First it is necessary to determine the direction in which a three dimensional volume needs to be projected, so that it can be lined up with the two dimensional image. For example it is possible to define a matrix of transformation by which an object can be transferred from the coordinate system of the three dimensional image into the two dimensional transillumination image. This enables the position and orientation of the three dimensional image to be adjusted so that its projection is brought into line with the two dimensional transillumination image. Image registration methods of this type are known from prior art and described for example in the article by J. Weese, T. M. Buzug, G. P. Penny, P. Desmedt: “2D/3D Registration and Motion Tracking for Surgical Interventions”, Philips Journal of Research 51 (1998), pages 299 to 316.

The second step involves the visualization of the registered images, that is, the combined display of the two dimensional image and the projected three dimensional image. Two standard methods are known among others for this purpose.

In a first method, known as “overlay”, the two images are placed one over the other as shown in FIG. 5. The share of the total combined image that each of the two individual images is intended to have can be adjusted. This is known in expert circles as “blending”.

In a second, less commonly used method known as “linked cursor”, the images are displayed in separate windows, both windows having a common cursor. Movements of a cursor or a catheter tip, for example, are transferred simultaneously into both windows.

The first method has the advantage that spatially linked pictorial information from different images is displayed visually at the same position. The disadvantage is that certain low contrast objects in the two dimensional image, including even catheter tips or stents, are covered over by the high contrast three dimensional recorded image on blending.

Although the second method does not have this problem, the surgeon has to work with two separate windows, providing less clarity during the operation and in some cases requiring a higher degree of caution. It is also more difficult to relate spatially linked pictorial information and image positions precisely, since they are visually separated.

U.S. Pat. No 6,317,621 B1 describes an example of a method for visualizing three dimensional objects, in particular in real time. This method first creates a three dimensional image data set of the object, for example from at least 2 two dimensional projection images, obtained by a C-arm X-ray device. Two dimensional transillumination images of the object are then recorded and registered with the three dimensional image data set. Visualization is carried out using “volume rendering”, wherein artificial light and shade effects are calculated, thus creating a three dimensional impression. Visualization can also be carried out by MIP (maximum intensity projection), although this rarely enables overlapping structures to be displayed.

A similar method is known from document U.S. Pat. No. 6,351,513 B1.

### SUMMARY OF THE INVENTION

The object of the present invention is to provide a method and a device for visualizing three dimensional objects, in particular in real time, whereby the objects can be viewed in a single window and even low contrast image areas can be seen with clarity.

This object is achieved by a method and by a device with the features which will emerge from the independent claims. Preferred embodiments of the invention are specified in the relevant dependent claims.

Advantageously, both in the inventive method and in the inventive device the two dimensional and three dimensional images are displayed together in one window, as in the overlay method, and their blending is preferably adjustable. However, the whole volume is not blended, but only lines that have been extracted from the object. Said lines may be those defining the outline of the object, for example. The lines preferably correspond in particular to the edges of the object, but can also define kinks, folds and cavities among other things. Furthermore the lines can also be extracted using more complex methods in order to show for example the center line of a tubular structure within the object. This can be performed with the aid of a filter that detects the second derivative of the gray levels in the image and thus captures the “burr” from the image. Alternatively or in addition to lines, points can also be extracted, defining for example the corners or other notable features of the object.

As a basic principle lines can be extracted and displayed in two different ways.

According to a first embodiment, the three dimensional image data set is projected (with correct perspective) only onto the image plane of the two dimensional transillumination image. The lines are then extracted from the projected volume and combined with the transillumination image. This method is suitable for extracting outlines, but in some circumstances spatial information about the object, such as edges, is lost during projection.

According to a second embodiment, lines are extracted from the three dimensional image data set by a suitable filter. These lines are then projected onto the image plane of the transillumination image and combined with said image. In this method it is possible to use for example a filter which generates a wire-mesh model of the object and extracts information such as edges or other lines from said model.

In both embodiments, the step in which lines are extracted from the object preferably has a step for binary encoding of the three dimensional data set or of the projected volume. Advantageously the edge pixels of the binary volume can easily be identified as the edges of the object.

Furthermore the step for extracting the object's lines from the three dimensional data set can have a step for the binary encoding of the object's volume and a step for projecting the encoded volume onto the image plane of the two dimensional transillumination image, the edge pixels of the projected binary volume defining the edges of the object.

Alternatively a standardized filter such as the known Prewitt, Sobel or Canny filters can also be used.

The three dimensional image data set of the object can preferably be created by fluoroscopic transillumination, computed tomography (CT), three dimensional angiography, three dimensional ultrasound, positron emission tomography (PET) or magnetic resonance tomography (MRT). If the chosen method is fluoroscopic transillumination, in which for example a three dimensional volume is reconstructed from a plurality of two dimensional images, it is then possible to use a C-arm X-ray device, which is also used for the subsequent surgical operation. This simplifies registration of the two dimensional images with the three dimensional image data set.

Preferably a step for adjustable blending of the object's lines onto the two dimensional transillumination images is provided in order to optimize the visualization. The actual blending can be very easily implemented and controlled with the aid of a joystick, which is also easy to maneuver during an operation.

### BRIEF DESCRIPTION OF THE DRAWINGS

Preferred embodiments of the invention are described below by reference to the accompanying drawings.

The drawings show:

FIG. 1 A view showing a three dimensional image of a heart, created by means of MRT;

FIG. 2 A view of a two dimensional transillumination image of said heart;

FIG. 3 A view of an inventive superimposition combining the two dimensional transillumination image with the edges of the three dimensional image of the heart;

FIG. 4 A diagram showing an X-ray device together with a device according to the present invention; and

FIG. 5 A view of a superimposition combining the two dimensional transillumination image with the three dimensional image according to prior art.

### DETAILED DESCRIPTION OF THE INVENTION

An exemplary embodiment of the invention will be described below by reference to the drawings.

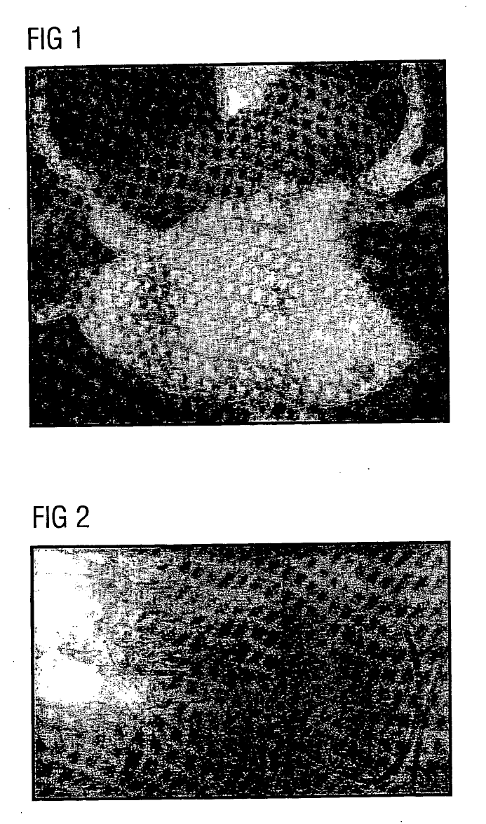

In the method according to the exemplary embodiment, a three dimensional image data set of the object is first created, said object being in this case a heart which is intended to be visualized. FIG. 1 shows a view of a three dimensional image of said heart, created by means of the magnetic resonance tomography method (MRT). Alternatively the three dimensional image can also be recorded by any method which enables the blood vessels or the structure of interest to be displayed with sufficient contrast, for example 3D angiography or 3D ultrasound. If the three dimensional image data set is intended to display other structures than blood vessels, the imaging method most suitable for the purpose in each case can be used, for example X-ray computer tomography (CT) or positron emission tomography (PET). Still further two dimensional images can be recorded by means of fluoroscopic transillumination and used to reconstruct a three dimensional image data set.

The three dimensional images are usually acquired before the actual surgical operation, for example on the previous day. If the chosen method for creating the three dimensional image data set is fluoroscopic transillumination, in which for example a three dimensional volume is reconstructed from a plurality of two dimensional images, it is then possible to use a C-arm X-ray device, which is also used for the subsequent surgical operation. This also simplifies registration of the two dimensional images with the three dimensional image data set.

The three dimensional image data set is stored on a data medium.

Two dimensional transillumination images of the heart are then recorded during the subsequent surgical operation, as shown in FIG. 2. In the case of the present exemplary embodiment, the two dimensional transillumination image of the heart is recorded by means of fluoroscopic X-ray transillumination in real time, which means for example that up to 15 recordings per second are made. This two dimensional transillumination image has no clear depth information and therefore shows no spatial details.

The three dimensional image data set is then registered with the two dimensional transillumination images, unless this was done at the same time as the three dimensional image data set was created. For example it is possible to define a matrix of transformation by which the object is transferred from the coordinate system of the three dimensional image into the two dimensional transillumination image. The position and orientation of the three dimensional image are adjusted so that its projection is brought into line with the two dimensional transillumination image.

In contrast to FIG. 2, FIG. 1 shows a view with depth information and spatial details. On the other hand, the three dimensional image according to FIG. 1 has a considerably higher contrast than the two dimensional transillumination image according to FIG. 2. If the two views are combined, the low contrast objects in the two dimensional transillumination image are covered by the high contrast objects in the MRT image and become almost invisible.

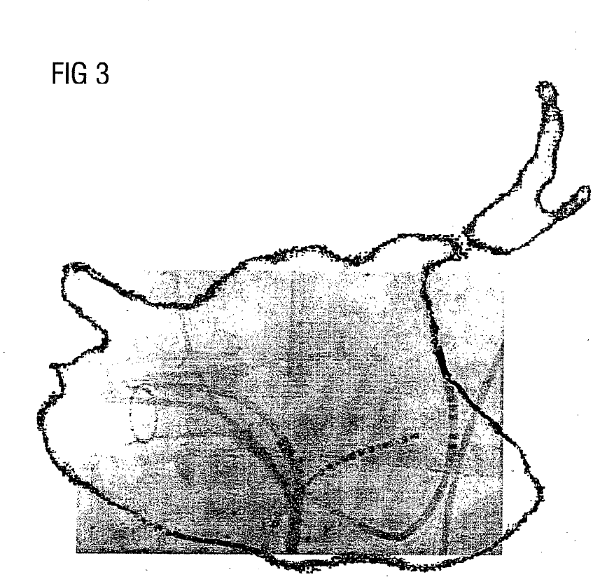

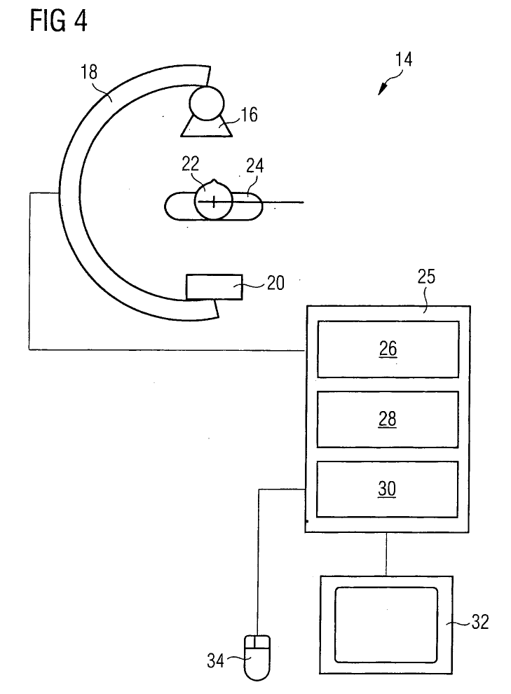

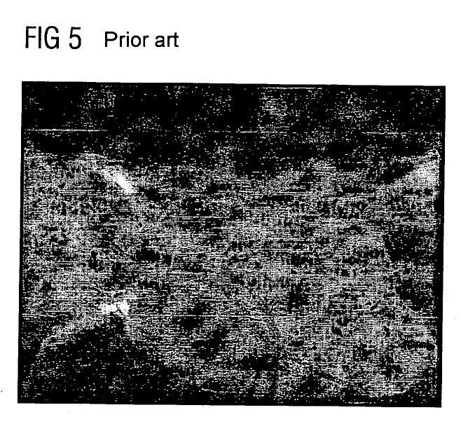

Therefore in the present invention the total volume of the three dimensional image is not superimposed, but only its external outlines. These lines are referred to below as “edges”, it being possible to use other types of lines such as center lines of blood vessels etc. The edges of the object are extracted from the three dimensional data set and visually combined with the two dimensional transillumination images, as shown in FIG. 3.

Extraction of the object's edges from the three dimensional data set can be implemented using different methods, wherein the edges define the outline of the object and can also include kinks, folds and cavities among other things.

Extraction of the object's edges from the three dimensional data set can preferably have a step for projecting the object's volume on the image plane of the two dimensional transillumination image and a step for the binary encoding of the projected volume. Advantageously the edge pixels of the binary volume can easily be defined by the edges of the object. Alternatively the step for extracting the object's edges from the three dimensional data set can have a step for the binary encoding of the object's volume and a step for projecting the encoded volume onto the image plane of the two dimensional transillumination image, the edge pixels of the projected binary volume defining the edges of the object.

Alternatively a standardized filter can also be used in order to extract the external edges of the object.

In the event that harsh color transitions in the image are emphasized while weak transitions are weakened further, a derivation filter or a Laplacian filter can be used.

Moreover non-linear filters such as a variance filter, extremal clamping filter, Roberts-Cross filter, Kirsch filter or gradient filter can also be used.

A Prewitt filter, Sobel filter or Canny filter can be implemented as the gradient filter.

A possible alternative is to make use of a method utilizing three dimensional geometrical grid models such as networks of triangles. The edges are projected into the two dimensional image, one of the adjacent surfaces of which points toward the camera and the other points away from the camera.

FIG. 4 shows an example of an X-ray device 14 which has a connected instrument that is used to create the fluoroscopic transillumination images. In the example shown, the X-ray device 14 is a C-arm device with a C-arm 18 having an X-ray tube 16 and an X-ray detector 20 attached to its arms. Said device could be for example the instrument known as Axiom Artis dFC from Siemens AG, Medical Solutions, Erlangen, Germany. The patient 24 is on a bed in the field of vision of the X-ray device. An object within the patient 24 is assigned the number 22, and is the intended target of the operation, for example the liver, heart or brain. Connected to the X-ray device is a computer 25. In the example shown, said computer not only controls the X-ray device but also handles the image processing. However, these two functions can also be performed separately. In the example shown, a control module 26 controls the movements of the C-arm and the recording of intraoperative X-ray images.

The preoperatively recorded three dimensional image data set is stored in a memory 28.

The three dimensional image data set is registered with the two dimensional transillumination images, recorded in real time, in a computing module 30.

Also in the computing module 30, the edges of the three dimensional object are extracted and combined with the two dimensional transillumination image. The combined image is displayed on a screen 32.

It is a simple matter for the user to blend the edges of the three dimensional object into the two dimensional transillumination image with the aid of a joystick or mouse 34, which is also easy to maneuver during an operation.

The present invention is not confined to the embodiments shown. Amendments to the scope of the invention defined by the accompanying claims are likewise included.

1-11. (canceled)

12. A method for visualizing a three dimensional object of a patient during a surgical intervention, comprising:
preoperatively recording a three dimensional image data set of the object;
recording a two dimensional transillumination image of the object;
registering the three dimensional image data set with the two dimensional transillumination image;
extracting a line of the object from the three dimensional image data set;
combining the two dimensional transillumination image with the extracted line of the object; and
displaying the two dimensional transillumination image combined with the extracted line.

13. The method as claimed in claim 12, wherein the extracting step comprises:
projecting the three dimensional image data set onto an image plane of the two dimensional transillumination image, and
extracting the line of the object by filtering the projected volume.

14. The method as claimed in claim 13, wherein the filtering comprises binary encoding the projected volume.

15. The method as claimed in claim 14, wherein pixels at an edge of the binary encoded volume is extracted as the line of the object that defines an edge of the object.

16. The method as claimed in claim 12, wherein the extracting step comprises:
extracting the line of the object by filtering the three dimensional image data set, and
projecting the extracted line onto an image plane of the two dimensional transillumination image.

17. The method as claimed in claim 16, wherein the filtering comprises binary encoding the three dimensional image data set.

18. The method as claimed in claim 17, wherein the binary encoded volume is projected onto the image plane of the two dimensional transillumination image.

19. The method as claimed in claim 18, wherein pixels at an edge of the binary encoded volume is extracted as the line of the object that defines an edge of the object.

20. The method as claimed in claim 12, wherein the three dimensional image data set of the object is recoded by a method selected from the group consisting of: fluoroscopic transillumination, computed tomography, three dimensional angiography, three dimensional ultrasound, positron emission tomography, and magnetic resonance tomography.

21. The method as claimed in claim 12, wherein the two dimensional transillumination image of the object is recorded in real time during the surgical intervention by a fluoroscopic transillumination.

22. The method as claimed in claim 12, wherein the three dimensional object of the patient is visualized in a real time during the surgical intervention.

23. The method as claimed in claim 12, wherein the line of the object is selected from the group consisting of: edge line of the object, outline of the object, and center line of the object.

24. The method as claimed in claim 12, wherein the line of the object is blended onto the two dimensional transillumination image.

25. A device for visualizing a three dimensional object of a patient during a surgical intervention, comprising:
an image recording device that records a two dimensional transillumination image of the object during the surgical intervention; and
a computer that:
registers a three dimensional image data set of the object with the two dimensional transillumination image,
extracts a line of the object from the three dimensional data set, and
combines the line of the object with the two dimensional transillumination image.

26. The device as claimed in claim 25, wherein the computer comprises a data memory that stores the three dimensional image data set of the object.

27. The device as claimed in claim 25, wherein the computer comprises a screen that displays the combined two dimensional transillumination image and the line of the object.

28. The device as claimed in one of the claims 25, wherein the computer blends the line of the object onto the two dimensional transillumination image.

29. The device as claimed in claim 25, wherein the image recording device is an X-ray image recording device.

30. The device as claimed in claim 25, wherein the line of the object defines an edge of the object.

31. The device as claimed in claim 25, wherein the three dimensional object is visualized in a real time during the surgical intervention.
